(19) United States
(12) Patent Application Publication
(10) Pub. No.: US 2003/0139396 A1
(43) Pub. Date: Jul. 24, 2003

(54) METHOD OF TREATMENT

(76) Inventor: Karen Gibson, Sheffield (GB)

Correspondence Address: ARTER & HADDEN, LLP, 1100 HUNTINGTON BUILDING, 925 EUCLID AVENUE, CLEVELAND, OH, 44115-1475, US

(21) Appl. No.: 10/108,659

(22) Filed: Mar. 27, 2002

Related U.S. Application Data
(63) Continuation-in-part of application No. 10/053,962, filed on Jan. 22, 2002.

(51) Int. Cl. A61K 31/5513

(52) U.S. Cl. 514/221

(57) ABSTRACT

There is described a method of treatment of a patient suffering from constipation characterised in that the method comprises the administration of a therapeutically effective amount of devazepide.

There is also described a method of treatment of a patient requiring analgesia which comprises the separate, simultaneous or sequential administration of a therapeutically effective amount of an analgesic and a stool softening amount of devazepide.

The use of devazepide in the manufacture of a medicament is also described.

This invention relates to a novel method of treatment related thereto and a novel use related thereto.

International Patent Application No. WO 99/18967 describes pharmaceutical compositions for treating chronic and neuropathic pain which comprises an analgesic amount of an opioid and an opioid potentiating amount of a CCK antagonist. WO '967 describes the use of both CCK-A antagonists and CCK-B antagonists, although it is described that, generally, CCK-B antagonists are preferred. Moreover, page 2, lines 6 to 8 of WO '967 describes that CCK-A antagonists may be suitable, but only at relatively higher dosages.

One specific CCK-A antagonist which is mentioned in WO 99/18967 is devazepide (Devacade®), which is 3s-(−)-1,3-dihydro-3-(2-indolecarbonylamino)-1-methyl-5-phenyl-2H-1,4-benzodiazepin-2-one.

Devazepide is commonly administered alongside an opioid analgesic, e.g. such as morphine. However, in normal doses, among the commonest side-effects of morphine and other opioid analgesics are nausea, vomiting, constipation, drowsiness, and confusion; tolerance generally develops with long-term use, but not to constipation which is the most common undesirable side effect of morphine treatment.

International Patent Application No. WO 99/18967 specifically describes a pharmaceutical formulation comprising a CCK antagonist, such as devazepide, an opioid and a biphasic carrier, comprising a glyceride derivative organic phase. This application suggests the possible use of a surfactant, especially when the formulation is in the form of an oil-in-water emulsion.

We have now surprisingly found that devazepide may also reduce or mitigate the undesirable side effects of opioid administration, e.g., in particular, constipation.

Thus, according to the invention we provide a method of treatment of a patient suffering from constipation characterised in that the method comprises the administration of a therapeutically effective amount of devazepide.

The method of the invention is especially suited to the treatment of constipation experienced due to the administration of an analgesic, such as an opioid analgesic.

Thus, according to the invention we especially provide a method of treatment of a patient suffering from constipation experienced due to the administration of an analgesic, such as an opioid analgesic, characterised in that the method comprises the administration of a therapeutically effective amount of devazepide.

The method of the invention may comprise the administration of an analgesic with devazepide, e.g. a stool softening amount of devazepide.

Thus, according to this aspect of the invention we provide a method of treatment of a patient requiring analgesia which comprises the separate, simultaneous or sequential administration of a therapeutically effective amount of an analgesic and a stool softening amount of devazepide.

Most preferably the amount of devazepide administered is sufficient so that the devazepide exhibits a stool softening effect and an analgesic potentiating effect.

In the method of the invention the analgesic is preferably an opioid. A variety of opioids may be used. Thus, the opioid may be selected from those which are effective analgesics and particularly those which need to be administered at relatively high or increasing doses. Examples include morphine, or a salt thereof such as the sulphate, chloride or hydrochloride, or the other 1,4-hydroxymorphinan opioid analgesics such as meperidine, butorphanol or pentazocine, or morphine-6-glucuronide, codeine, dihydrocodeine, diamorphine, dextropropoxyphene, pethidine, fentanyl, alfentanil, alphaprodine, buprenorphine, dextromoramide, diphenoxylate, dipipanone, heroin (diacetylmorphine), hydrocodone (dihydrocodeinone), hydromorphone (dihydromorphinone), levorphanol, meptazinol, methadone, metopon (methyldihydromorphinone), nalbuphine, oxycodone (dihydrohydroxycodeinone), oxymorphone (dihydrohydroxymorphinone), phenadoxone, phenazocine, remifentanil, tramadol, or a salt of any of these. Naloxone is also included within the definition of an opioid. Especially preferred analgesics which may be mentioned are hydromorphone, oxycodone, morphine, e.g. morphine sulphate and fentanyl. In a preferred embodiment of the invention the analgesic is morphine or morphine sulphate. In a further preferred embodiment the opioid is fentanyl, or a salt thereof.

In the method of the invention the devazepide and/or the opioid may be administered using any methods conventionally known per se. Thus, such methods would include, but shall not be limited to, administration intravenously, orally, intrathecally, intranasally, intrarectally, intramuscularly/subcutaneously, by inhalation and by transdermal patch. When the devazepide and/or opioid is administered intravenously, it may, for example, be as an intravenous bolus or a continuous intravenous infusion. When the devazepide and/or the opioid is administered subcutaneously, it may, for example, be by subcutaneous infusion. Preferably, the opioid and/or devazepide are administered orally. Preferentially, the opioid and the devazepide will be administered using the same mode of administration. Thus, for example, when the opioid is administered orally then the devazepide may be administered orally also. However, it is within the scope of the invention for the opioid to be administered intravenously and the devazepide to be administered orally. In a further preferred embodiment the opioid may be administered by a transdermal patch. When a transdermal patch is used, the preferred opioid is fentanyl.

Thus, in the method of the invention the daily dosage of devazepide may vary depending upon, inter alia, the weight of the patient, the method of administration, etc. In patients that are suffering serious disorders, such as cancer patients, the weight of the patient may be very low and therefore the dosage of devazepide consequentially may be low. Thus the daily dosage of devazepide may be up to 0.7 mg/kg/day. Preferably, the daily dosage of devazepide may be from 25 μg/kg/day to 0.7 mg/kg/day, more preferably from 50 μg/kg/day to 0.5 mg/kg/day. For oral administration the daily dosage of devazepide may be from 0.07 mg/kg/day to 0.7 mg/kg/day, preferably 0.07 mg/kg/day to 0.29 mg/kg/day. For intravenous administration the dosage of devazepide is preferably 50 μg/kg/day to 0.5 mg/kg/day.

In the method of the invention the dosage of the opioid analgesic administered may vary depending upon, inter alia, the nature of the opioid analgesic, the weight of the patient, the method of administration, etc. Thus, for example, the dosage of, e.g. an opioid, such as morphine, may be from 5 to 200 mg daily. A particular dosage which may be mentioned is from 10 to 240 mg daily. A daily dosage of morphine may be from 5 to 100 mg or occasionally up to 500 mg.

In the method of the invention devazepide may be provided as a composition incorporating, for example, a filler, or other excipient.

Thus, for example, in one embodiment of the invention the composition may be made up into a capsule formulation, e.g. with a fill weight of 150 mg±5% by weight or 300 mg±5% by weight. In the one preferred embodiment, the capsule formulation may comprise 1.25 mg devazepide, and 148.75 mg of a filler or other excipient, e.g. corn starch. In a further preferred embodiment, the capsule formulation may comprise 2.5 mg devazepide, and 297.5 mg of a filler or other excipient, e.g. corn starch.

Thus, such fillers may be selected from the group lactose, mannitol, talc, magnesium stearate, sodium chloride, potassium chloride, citric acid, spray-dried lactose, hydrolysed starches, directly compressible starch, microcrystalline cellulose, cellulosics, sorbitol, sucrose, sucrose-based materials, calcium sulphate, dibasic calcium phosphate and dextrose. A preferred filler is starch, e.g. corn starch.

When the composition of the invention includes a filler, the size of the devazepide and filler particles may be the same or different. However, in a preferred embodiment the sizes of the devazepide and filler particles will differ. Preferentially, the devazepide and/or the filler may be of reduced particle size, e.g. by milling.

A preferred embodiment of the invention comprises a formulation as hereinbefore described filled into a capsule. Any conventionally known materials may be used for the capsule, however a preferred material is gelatin.

According to a further aspect of the invention we provide a method of treatment of a patient undergoing opioid analgesic therapy which comprises the administration of a pharmaceutical composition as hereinbefore described.

According to a further aspect of the invention we provide the use of devazepide in the manufacture of medicament suitable for use in a method as hereinbefore described.

The devazepide used in the method of the invention is the S enantiomer, preferentially, the S enantiomer wherein the level of R enantiomer, which may be present as an impurity, is not greater than 1.5% w/w.

1. A method of treatment of a patient suffering from constipation characterised in that the method comprises the administration of a therapeutically effective amount of devazepide.

2. A method of treatment according to claim 1 characterised in that the constipation experienced is due to the administration of an analgesic.

3. A method of treatment according to claim 2 characterised in that the analgesic is an opioid analgesic.

4. A method of treatment according to claim 1 characterised in that the method comprises the administration of an analgesic with a stool softening amount of devazepide.

5. A method of treatment of a patient requiring analgesia which comprises the separate, simultaneous or sequential administration of a therapeutically effective amount of an analgesic and a stool softening amount of devazepide.

6. A method of treatment according to any one of claims 1 or 5 characterised in that the amount of devazepide administered is sufficient so that the devazepide exhibits a stool softening effect and an analgesic potentiating effect.

7. A method of treatment according to claim 5 characterised in that the analgesic is an opioid analgesic.

8. A method according to any one of claims 3 or 7 characterised in that the opioid is selected from morphine, or a salt thereof such as the sulphate, chloride or hydrochloride, or the other 1,4-hydroxymorphinan opioid analgesics such as meperidine, butorphanol or pentazocine, or morphine-6-glucuronide, codeine, dihydrocodeine, diamorphine, dextropropoxyphene, pethidine, fentanyl, alfentanil, alphaprodine, buprenorphine, dextromoramide, diphenoxylate, dipipanone, heroin (diacetylmorphine), hydrocodone (dihydrocodeinone), hydromorphone (dihydromorphinone), levorphanol, meptazinol, methadone, metopon (methyldihydromorphinone), nalbuphine, oxycodone (dihydrohydroxycodeinone), oxymorphone (dihydrohydroxymorphinone), phenadoxone, phenazocine, remifentanil, tramadol, or a salt of any of these.

9. A method according to any one of claims 3 or 7 characterised in that the opioid is naloxone.

10. A method according to claim 8 characterised in that the analgesic is selected from the group hydromorphone, oxycodone, morphine, e.g. morphine sulphate and fentanyl.

11. A method according to claim 8 characterised in that the opioid is fentanyl or a salt thereof.

12. A method according to claim 10 characterised in that the analgesic is selected from the group morphine and morphine sulphate.

13. A method according to any one of claims 1 or 5 characterised in that the devazepide and/or the opioid may be administered using a method selected from administration intravenously, orally, intrathecally, intranasally, intrarectally, intramuscularly/subcutaneously, by inhalation and by transdermal patch.

14. A method according to claim 13 characterised in that the devazepide and/or the opioid is administered intravenously.

15. A method according to claim 14 characterised in that the intravenous administration is by intravenous bolus or a continuous intravenous infusion.

16. A method according to claim 13 characterised in that the devazepide and/or the opioid is administered subcutaneously.

17. A method according to claim 16 characterised in that the subcutaneous administration is as a subcutaneous infusion.

18 A method according to claim 13 characterised in that the opioid and/or devazepide are administered orally.

19. A method according to claim 13 characterised in that the opioid and the devazepide are administered using the same mode of administration.

20. A method according to claim 19 characterised in that the opioid is administered orally and the devazepide is administered orally.

21. A method according to claim 13 characterised in that the opioid is administered by transdermal patch.

22. A method according to claim 21 characterised in that the opioid is fentanyl, or a salt thereof.

23. A method according to any one of claims 1 or 5 characterised in that the daily dosage of devazepide may be up to 0.7 mg/kg/day.

24. A method according to claim 23 characterised in that the daily dosage of devazepide is from 25 μg/kg/day to 0.7 mg/kg/day.

25. A method according to claim 24 characterised in that the daily dosage of devazepide is from 50 μg/kg/day to 0.5 mg/kg/day.

26. A method according to claim 23 characterised in that the dosage of devazepide is an oral dosage.

27. A method according to claim 26 characterised in that for oral administration the daily dosage of devazepide is from 0.07 mg/kg/day to 0.29 mg/kg/day.

28. A method according to claim 14 characterised in that for intravenous administration the dosage of devazepide is 50 μg/kg/day to 0.5 mg/kg/day.

29. A method according to claim 3 or 5 characterised in that the dosage of the opioid is from 5 to 2000 mg daily.

30. The use of devazepide in the manufacture of medicament suitable for use as a stool softener.

31. The use according to claim 30 characterised in that the medicament is suitable for co-administration comprises the separate, simultaneous or sequential administration with an opioid analgesic.

32. The use according to claim 31 characterised in that the medicament comprises a composition of devazepide and an opioid analgesic.

33. The use according to claim 30 characterised in that the devazepide is provided as a composition incorporating a filler or other excipient.

34. The use according to claim 33 characterised in that the composition is filled into a capsule.

35. The use according to claim 34 characterised in that the capsule is gelatin capsule.

36. The use according to claim 34 characterised in that the composition is made up into a capsule formulation with a fill weight of 150 mg±5% by weight or 300 mg±5% by weight.

37. The use according to claim 34 characterised in the capsule comprises 1.25 mg devazepide or 2.5 mg devazepide.

38. A method of treatment of a patient undergoing opioid analgesic therapy which comprises the administration of a capsule formulation comprising 1.25 mg devazepide or 2.5 mg devazepide.

39. A method according to claims 1 or 5 characterised in that the devazepide used in the method of the invention is substantially the S enantiomer.

40. A method according to claim 39 characterised in that the level of R enantiomer, which may be present as an impurity, is not greater than 1.5% w/w.

41. A composition or a method substantially as described with reference to the accompanying examples.
